(19) United States
(12) Patent Application Publication
(10) Pub. No.: US 2026/0102636 A1
(43) Pub. Date: Apr. 16, 2026

(54) SYSTEMS, DEVICES, AND METHODS FOR USING FOCUSED ULTRASONIC ENERGY TO INHIBIT COMMUNICATION OF PERIPHERAL SENSORY NERVES

(71) Applicant: THE BOARD OF TRUSTEES OF THE LELAND STANFORD JUNIOR UNIVERSITY, Stanford, CA (US)

(72) Inventors: Thomas Anthony Anderson, Menlo Park, CA (US); Kim Butts-Pauly, Stanford, CA (US); David Yeomans, Stanford, CA (US)

(21) Appl. No.: 19/422,193

(22) Filed: Dec. 16, 2025

Related U.S. Application Data
(63) Continuation of application No. PCT/US24/35124, filed on Jun. 21, 2024.
Application No. PCT/US24/35124 is a continuation-in-part of application No. 18/542,494, filed on Dec. 15, 2023.
Application No. 18/542,494 is a continuation of application No. PCT/US2022/033687, filed on Jun. 15, 2022.
(60) Provisional application No. 63/210,564, filed on Jun. 15, 2021.
(60) Provisional application No. 63/314,143, filed on Feb. 25, 2022.
(60) Provisional application No. 63/522,319, filed on Jun. 21, 2023.

(51) Int. Cl. A61N 7/00

(52) U.S. Cl. CPC A61N 7/00; A61N 2007/0026

(57) ABSTRACT

Devices, systems, and methods are provided for delivering focused ultrasound into a subject's body, e.g., by exposing a target peripheral nerve to focused ultrasonic energy to modulate the target nerve and disrupt a communication pathway between the target nerve and a subject's central nervous system, e.g., for relieving acute or chronic pain.

### RELATED APPLICATION DATA

The present application is a continuation of co-pending International Application No. PCT/US2024/035124, filed Jun. 21, 2024, which claims benefit of U.S. provisional application Ser. No. 63/522,319, filed Jun. 21, 2023, and is a continuation-in-part of co-pending U.S. application Ser. No. 18/542,494, filed Dec. 15, 2023, which is a continuation of International Application No. PCT/US2022/033687, filed Jun. 15, 2022, which claims benefit of U.S. provisional applications Ser. Nos. 63/210,864, filed Jun. 15, 2021, and 63/314,143, filed Feb. 25, 2022, the entire disclosures of which are expressly incorporated by reference herein.

### TECHNICAL FIELD

The present application relates to medical devices and, more particularly, to devices, systems, and methods for delivering focused ultrasound into a subject's body, e.g., by exposing a target peripheral nerve to focused ultrasonic energy to modulate the target nerve and disrupt a communication pathway between the target nerve and a subject's central nervous system, e.g., for relieving acute or chronic pain.

### BACKGROUND

Moderate-to-severe acute pain is prevalent in many healthcare settings and associated with adverse outcomes. For example, a patient may experience acute pain, e.g., due to surgery or an injury, or the pain may be chronic pain, e.g., due to conditions such as complex regional pain syndrome (CRPS) type 1 and 2, phantom limb pain, trigeminal neuralgia, Bell's palsy, intercostal pain, post-herpatic neuralgia, endometriosis, and neuroma.

The most common method for pain management is narcotics, which may have a range of adverse side effects. For example, peripheral nerve blockade using traditional needle-based and local anesthetic based techniques improves pain outcomes for some patient populations but has shortcomings limiting use. These limitations include its invasiveness, potential for local anesthetic systemic toxicity, risk of infection with an indwelling catheter, and relatively short duration of blockade compared with the period of pain after major injuries.

Accordingly, devices and methods for improving pain management, e.g., for non-drug-based pain suppression, would be useful.

### SUMMARY

The present application is directed to medical devices and, more particularly, to devices, systems, and methods for delivering focused ultrasound into a subject's body. For example, the devices systems, and methods herein may involve exposing a target peripheral nerve to focused ultrasonic energy to modulate the target nerve and disrupt a communication pathway between the target nerve and a subject's central nervous system, e.g., for relieving acute or chronic pain.

In accordance with one example, a system is provided for modulating peripheral sensory nerves and/or peripheral sensory nerve fibers in a subject's body that includes an imaging device configured to identify a target nerve within the subject's body; a housing including a surface configured for placement against the subject's skin; one or more transducer elements carried by the housing configured to deliver focused ultrasound from the surface into the subject's body; and a controller coupled to the imaging device to identify the target nerve and coupled to the one or more transducer elements to control delivery of the focused ultrasound to the target nerve.

In accordance with another example, a system is provided for modulating peripheral sensory nerves and/or peripheral sensory nerve fibers in a subject's body that includes a housing including a surface configured for placement against the subject's skin; one or more transducer elements carried by the housing configured to deliver focused ultrasound from the surface into the subject's body; an imaging device mounted on the housing adjacent the surface and configured to acquire images within the subject's body; and a controller coupled to the imaging device and the one or more transducer elements, wherein the controller is configured to: a) analyze information regarding a target nerve based at least in part on images acquired by the imaging device; b) determine one or more parameters of the focused ultrasound based at least in part on a target degree of neuromodulation of the target nerve; c) calibrate the one or more transducers to deliver focused ultrasound based on the one or more parameters; and d) activate the one or more transducers to deliver the focused ultrasound to the target nerve. Optionally, the controller is further configured to: e) deactivate the one or more transducers to discontinue delivery of the focused ultrasound; f) determine whether the target degree of neuromodulation has been achieved; and if additional focused ultrasound is needed to achieve the target degree of neuromodulation, repeating steps b) to e).

In accordance with still another example, a system is provided for modulating peripheral sensory nerves and/or peripheral sensory nerve fibers in a subject's body that includes an imaging device configured to identify a plurality of target nerves within the subject's body; a housing including a surface configured for placement against the subject's skin; a plurality of transducer elements carried by the housing configured to deliver focused ultrasound from the surface into the subject's body; and a controller coupled to the imaging device to identify the target nerves and coupled to the transducer elements to control delivery of the focused ultrasound to the target nerves to focus the transducer elements to deliver focused ultrasound simultaneously to the plurality of target nerves.

In accordance with still another example, a system is provided for modulating peripheral sensory nerves and/or peripheral sensory nerve fibers in a subject's body that includes an imaging device configured to identify a target nerve within the subject's body; a first delivery device comprising a first housing including a surface configured for placement against the subject's skin, and one or more transducer elements carried by the first housing configured to deliver focused ultrasound from the surface into the subject's body; a second delivery device comprising a second housing including a surface configured for placement against the subject's skin adjacent the first housing, and one or more transducer elements carried by the second housing configured to deliver focused ultrasound from the surface into the subject's body; and a controller coupled to the imaging device to identify the target nerve and coupled to the one or more transducer elements of the first and second delivery devices to control delivery of the focused ultrasound to the target nerve.

In accordance with another example, a method is provided for modulating peripheral sensory nerves and/or peripheral sensory nerve fibers in a subject's body that includes placing a surface of a housing against the subject's skin to position one or more transducer elements carried by the housing to deliver focused ultrasound from the surface into the subject's body; acquiring images of locations within the subject's body using an imaging device mounted on the housing; analyzing information regarding the target nerve based at least in part on images acquired by the imaging device; determining one or more parameters of the focused ultrasound based at least in part on a target degree of neuromodulation of the target nerve; calibrating the one or more transducers to deliver focused ultrasound based on the one or more parameters; and activating the one or more transducers to deliver the focused ultrasound to the target nerve.

Other aspects and features of the present invention will become apparent from consideration of the following description taken in conjunction with the accompanying drawings.

### BRIEF DESCRIPTION OF THE DRAWINGS

It is believed the present invention will be better understood from the following description of certain examples taken in conjunction with the accompanying drawings, in which like reference numerals identify the same elements and in which:

FIG. 1 is a block diagram of an exemplary system for executing one or more methods disclosed herein to calibrate and/or use a FUS device to modulate a target nerve.

FIG. 1A is a cross-sectional of an exemplary device for delivering focused ultrasound into a subject's body that may be included in the system of FIG. 1.

FIG. 1B is a cross-sectional of another exemplary device for delivering focused ultrasound into a subject's body that may be included in the system of FIG. 1.

FIG. 2A is a block diagram of a first exemplary FUS delivery device that may be included in the system of FIG. 1.

FIG. 2B is a block diagram of a second exemplary FUS delivery device that may be included in the system of FIG. 1.

FIG. 3 is a flowchart illustrating an exemplary process for delivering FUS to a target nerve within a subject.

FIG. 4 is an exemplary ultrasound image of a subject's tissue.

FIG. 5A is a two-dimensional schematic diagram showing an exemplary cross-section of subject tissue that includes a target nerve being treated with FUS that has a relatively narrow vertically oriented focal zone emanating from a single FUS transducer.

FIG. 5B is a two-dimensional schematic diagram showing another exemplary cross-section of subject tissue that includes a target nerve being treated with FUS that has a relatively wide vertically oriented focal zone emanating from a single FUS transducer.

FIG. 5C is a two-dimensional schematic diagram showing yet another exemplary cross-section of subject tissue that includes a target nerve being treated with FUS that has a relatively wide horizontally oriented focal zone emanating from a single FUS transducer.

FIG. 6A is a two-dimensional schematic diagram showing another exemplary cross-section of subject tissue that includes a target nerve being treated with two beams of FUS emanating from two separate FUS transducers.

FIG. 6B is a two-dimensional schematic diagram showing still another cross-section of subject tissue that includes a flat and wide target nerve being treated with two beams of FUS emanating from two separate FUS transducers.

FIG. 6C is a two-dimensional schematic diagram showing another cross-section of subject tissue that includes a first target nerve being treated with a first beam of FUS emanating from a first FUS transducer and a second target nerve being treated with a second beam of FUS emanating from a second FUS transducer.

FIG. 6D is a two-dimensional schematic diagram showing another cross-section of subject tissue that includes a first target nerve being treated with a first beam of FUS emanating from a first FUS transducer and a second target nerve being treated with a second beam of FUS emanating from a second FUS transducer, wherein the first target nerve is positioned closer to the subject's skin than the second target nerve.

FIG. 7A is a cross section view of the system of FIG. 2B illustrating an example of a plurality of the FUS transducers is projecting FUS toward two target nerves of similar size and shape.

FIG. 7B is a cross section view of the system of FIG. 2B illustrating another example of a plurality of FUS transducers projecting FUS toward two target nerves of different size and shape.

FIG. 7C is a cross section view of the system of FIG. 2B illustrating another example of a plurality of FUS transducers projecting FUS toward two target nerves, wherein a second of the target nerve is closer to the skin than a first target nerve.

FIG. 8 is a flowchart illustrating a process for treating a target nerve of a subject using FUS.

The drawings are not intended to be limiting in any way, and it is contemplated that various examples of the invention may be carried out in a variety of other ways, including those not necessarily depicted in the drawings. The accompanying drawings incorporated in and forming a part of the specification illustrate several aspects of the present invention, and together with the description serve to explain the principles of the invention; it being understood, however, that this invention is not limited to the precise arrangements shown.

### DETAILED DESCRIPTION

The following description of certain examples of the invention should not be used to limit the scope of the present invention. Other examples, features, aspects, embodiments, and advantages of the invention will become apparent to those skilled in the art from the following description, which is by way of illustration, one of the best modes contemplated for carrying out the invention. As will be realized, the invention is capable of other different and obvious aspects, all without departing from the invention. Accordingly, the drawings and descriptions should be regarded as illustrative in nature and not restrictive.

Before the examples are described, it is to be understood that the invention is not limited to particular examples described, as such may, of course, vary. It is also to be understood that the terminology used herein is for the purpose of describing particular examples only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, some potential and exemplary methods and materials are now described.

It must be noted that as used herein and in the appended claims, the singular forms “a,” “an,” and “the” include plural referents unless the context clearly dictates otherwise. Thus, for example, reference to “a compound” includes a plurality of such compounds and reference to “the polymer” includes reference to one or more polymers and equivalents thereof known to those skilled in the art, and so forth.

Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number may be a number which, in the context in which it is presented, provides the substantial equivalent of the specifically recited number.

In various examples, the present application is directed to exposing a target peripheral nerve to focused ultrasonic energy (FUS) to modulate the target nerve and disrupt a communication pathway between the target nerve and a subject's central nervous system by exciting and/or suppressing sensory nerve conduction so that the subject temporarily (e.g., for about one hour to twelve weeks), or permanently, no longer perceives a sensation of pain germinating from the treated target nerve. In many cases, the FUS may be tailored and/or delivered so that non-pain sensory and motor functions of the target nerve are not impacted or are only minimally impacted (i.e., less impacted than the pain function) so that, for example, the target nerve may still enable the subject to experience non-pain sensation and perform motor functions. The systems, devices, and/or methods disclosed herein may be used in situations where a subject is currently experiencing (e.g., following a traumatic injury), and/or is expected to experience (e.g., surgery) acute pain to decrease and/or eliminate the subject's perception of pain germinating from the target nerve. The systems, devices and methods disclosed herein may also be used to treat chronic pain by, for example, ablating one or more target nerves and/or inducing neurolysis therein.

In some examples, a target nerve may be identified, characterized (e.g., type, size, shape, etc.), and/or located within a subject using one or more images thereof and the FUS delivered to the target nerve may be tuned or adjusted based on interpretation and/or analysis of the image(s) to achieve a desired effect such as a desired degree of neuromodulation caused by the applied FUS, which may correspond to a desired degree and/or duration of communication disruption and/or pain blockade for the treated, target nerve. For example, one or more characteristics (e.g., intensity, frequency, focal zone geometry, etc.) of the FUS may be tunable to achieve a disruption of pain sensation communication from a target nerve for a desired length of time (e.g., about one to twenty four hours, one to six days, or one to twelve weeks). Following treatment with FUS, the target nerve may recover (i.e., regain an ability to sense pain and/or communicate sensations of pain to a subject's nervous system) over time via neuroregeneration according to a natural and/or accelerated (e.g., using pharmaceuticals or other treatments) healing process.

In various examples, the systems and/or devices disclosed herein may be configured to be noninvasively used. Calibration (e.g., determining a focal distance, frequency, and/or intensity) and/or positioning of a FUS transducer so that the FUS may be properly applied to one or more target nerves may be achieved, in some cases, using image guidance as may be provided, for example, via ultrasound and/or MRI imaging techniques. At times, FUS transducer location and/or calibration may be determined before or during use (e.g., by application of the transducer to the subject's epidermis). Once the correct position and focal distance are determined, the FUS transducer may be directly applied onto the epidermis with or without coupling material (e.g., gel) and/or a pad between the transducer and epidermis and focused ultrasonic energy may be transmitted to the target nerve through the subject's skin and tissue.

As used herein, the term “target nerve” may correspond to a peripheral nerve, one or more locations along a peripheral nerve (including the nerve roots, trunks, divisions, and cords), a group of peripheral nerves, a nerve plexus, and/or a group of nerve plexuses. In some examples, one or more a target nerves may be treated with FUS to eliminate, or limit, sensations of pain for one or more areas (e.g., a limb, torso, joint, etc.) of a subject's body.

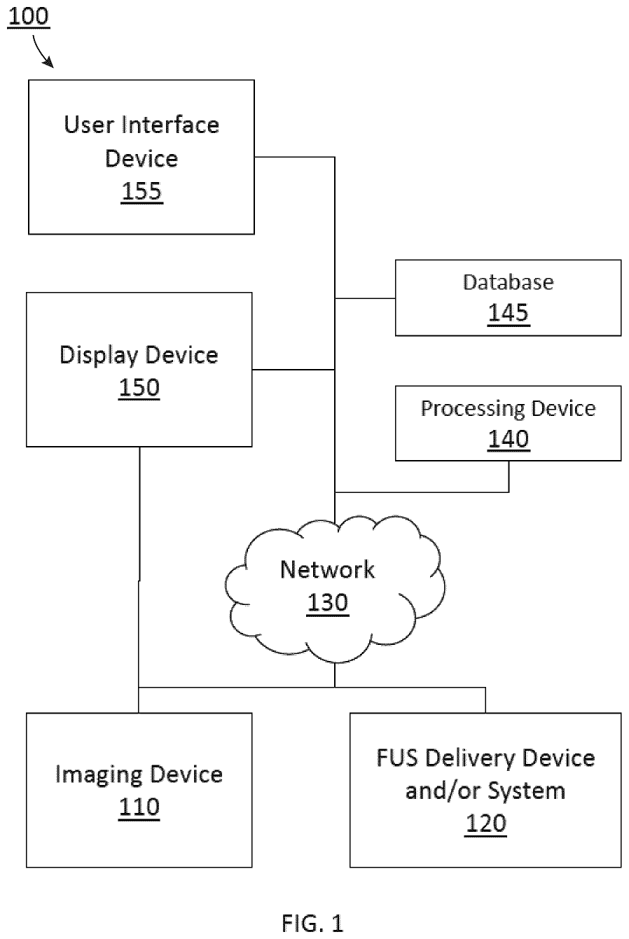

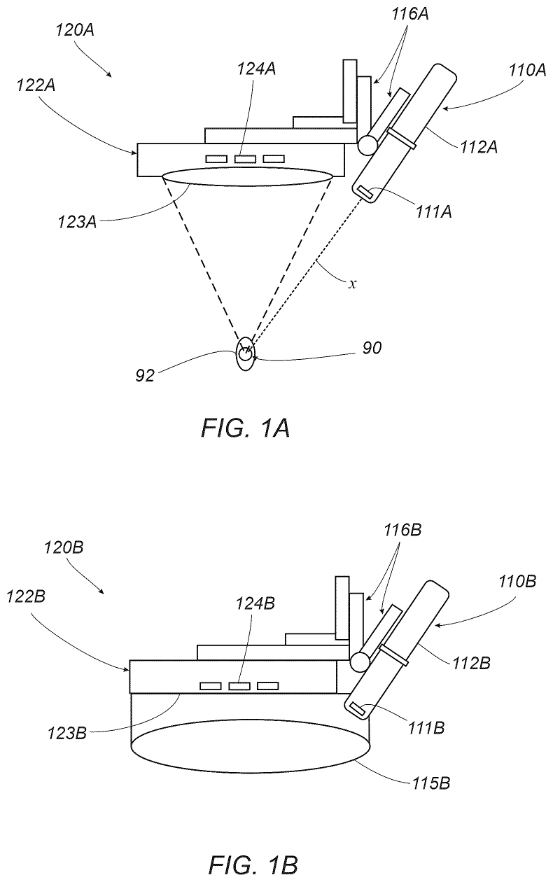

Turning now to the drawings, FIG. 1 is a block diagram of a system 100 for executing one or more methods disclosed herein, for example, to calibrate and/or use a FUS device to modulate a target nerve such as the target nerves discussed herein. The system 100 includes an imaging device 110, a FUS delivery device/system 120, a user interface device 155, a network 130, a processing device 140, a database 145, and a display device 150. Although shown as separate components, some components of the system 100 may be resident within the same housing. For example, the processing device 140, database 145, user interface 155, and display device 150 may all be resident in one device, such as a computer or a terminal. Additionally, or alternatively, the imaging device 110 and FUS delivery device/system 120 may be resident in the same housing. Communication between two or more components of the system 100 may be wired and/or wireless and, in some instances, may be facilitated by a network 130, which may be a private communication network and/or the Internet. In some examples, one or more components of the system 100 (e.g., the processing device 140 and/or database 145) may be resident within a cloud computing environment.

The user interface device 155 may be any device, or set of devices, that a user may employ to interact with one or more components of the system 100. Exemplary user interface devices 155 include, but are not limited to, keyboards, trackpads, touchscreens, speakers, and/or microphones. The processing device 140 may be any device programmed with software or instructions to execute one or more methods disclosed herein and/or operate one or more components of system 100. Exemplary processing devices 140 include, but are not limited to, processors, field programmable gate arrays (FPGAs), and/or application specific integrated circuits (ASIC). The database 145 may be configured, for example, to store one or more sets of instructions for execution by the processing device 140, imaging device 110, and/or FUS delivery device/system 120. In some examples, the database 145 may include information about one or more subjects who may receive treatment using the system 100, for example, as an electronic medical record (EMR). The display device 150 may be any device configured to display information, instructions, and/or images to a user in, for example, accordance with one or more methods disclosed herein.

The imaging device 110 may be any device configured to image subcutaneous tissue (e.g., adipose, muscle, and/or nerve) of a subject. Exemplary imaging devices 110 include ultrasound imaging devices, MRI devices, and CT scanners. The FUS delivery device/system 120 may be any device or set of devices configured to project focused ultrasonic energy into the tissue of a subject toward a target nerve such as sensory nerve 510, for example in accordance with one or more methods disclosed herein. In some examples, the FUS delivery device/system 120 may be configured to deliver focused low-intensity ultrasonic energy, such as ultrasonic energy with an exemplary intensity ranging between about zero and five hundred Watts per square centimeter (0-500 W/cm²) and/or an exemplary frequency within a range of about half to two Megahertz (0.5-2.0 MHz) (e.g., about 1.5 MHz) to targets structures (e.g., sensory nerve 510). Additionally or alternatively, the FUS delivery device/system 120 may be configured to deliver ultrasonic energy at frequencies within a range of about one kilohertz and ten Megahertz (1 kHz-10 MHz), e.g., with an exemplary intensity ranging between about zero and five hundred Watts per square centimeter (0-500 W/cm²). Additionally, or alternatively, the FUS delivery device/system 120 may be configured to deliver relatively high-intensity ultrasonic energy with frequencies within a range of about one kilohertz and ten Megahertz (1 kHz-10 MHz) and an intensity within a range of about five hundred to two thousand Watts per square centimeter (500-2000 W/cm²).

FIG. 1A shows an example of a delivery device 120A that may be included in the system 100 of FIG. 1. Generally, the device 120A includes a housing 122A containing or carrying one or more components, e.g., an array of transducers 124A and an imaging or locating element 110A. The housing 122A may include a contact surface 123A configured for placement against the subject's skin (not shown), e.g., to allow focused ultrasound to be delivered from transducers 124A through the surface 123A into the subject's body to a target nerve 90, as described further elsewhere herein. Alternatively, as shown in FIG. 1B, a balloon, pad, or other transition member 115B may be provided on the housing 122B, e.g., attached to the surface 123B. As described elsewhere herein, the transition member 115B may facilitate acoustically coupling the housing 124B and, consequently, the transducers 124B, to the subject's skin. Optionally, the transition member may be an inflatable balloon or bladder, e.g., may include an interior cavity that may be selectively filled with media, e.g., water, gel, and the like, to adjust a size and/or shape of the transition member, e.g., to conform to the subject's skin and/or position the transducers at a desired distance from the subject's skin. The membrane may be formed from inelastic material such that the transition member has a substantially fixed size when filled or may be formed from elastic material to allow the size of the transition member to be adjusted based on the volume of fluid introduced into the interior cavity.

Optionally, the housing 122A (or 122B) may be shaped to provide a handheld device, e.g., having a shape to facilitate the subject holding the device 120A. For example, the housing 122A itself may be shaped to allow a user to hold the device 120A, e.g., having an elongated cylindrical or other shape, and/or one or more handles, grips, or other features (not shown) may extend from the housing 122A to facilitate manipulation of the device 10 during use. For example, the transducers 124A may be provided on a head of a wand, e.g., having a disk or other shape corresponding to the shape of the transducers including a flat, concave, or convex contact surface for placement against the subject's skin.

Alternatively, the housing 122A (or 122B) may be mounted to an arm or other structure (not shown) that allow the device 120A to be manipulated and supported from a location adjacent the subject being treated. For example, a cart may be provided that includes various components of the system 100, e.g., a controller box and display (not shown), and an arm may be mounted to the cart that carries the device 120A. The arm may include one or more joints, which allows the location of the device 120A to be adjusted relative to the cart and subject, e.g., to allow the contact surface 123A or transition member 115B to be placed against the subject's body while remaining supported by the arm and cart.

In addition or alternatively, the housing 122A, 122B may include one or more features to facilitate securing the device 120A, 120B relative to the subject's body. For example, the contact surface 123A or transition member 115B may include adhesive or other tacky material that may secure the surface 123A or transition member 115B to the subject's skin during use, but allow the device 120A, 120B to be removed without damaging the skin. In addition or alternatively, one or more straps (not shown) may be provided on the housing 122A, 122B that may be wrapped at least partially around the subject's body, e.g., a pair of straps on opposite sides of the contact surface 123A having sufficient length to allow the straps to be wrapped around an appendage, e.g., arm or leg, or the torso of the subject's body during use. Optionally, the straps may include ends with cooperating fasteners, e.g., hook and eye fasteners, snaps, clips, buttons, ties, and the like (not shown), for removably securing the ends together to hold the contact surface 123A or transition member 115B against the skin. Alternatively, the subject or another person may simply hold the housing 122A, 122B and press the surface 123A or transition member 115B against the skin during use.

With continued reference to FIG. 1A, in one example, the imaging or locating element 110A includes an imaging or diagnostic ultrasound transducer 111A configured to transmit incident ultrasound signals, e.g., into the subject's body, and receive reflected signals from the body, e.g., centered on an axis “x.” The imaging element 110A may be mounted to the housing 122A such that the imaging transducer 111A is offset from the FUS transducers 124A, e.g., laterally relative to the contact surface 123A, and angled such that the axis “x” intersects a central axis of the focused ultrasound delivered by the transducers 124A. The imaging transducer 111A may include one or more piezoelectric elements (one shown for simplicity), e.g., provided at a distal end of an imaging housing 112A mounted adjacent the transducers 124A such that the imaging housing 112A contacts the subject's skin adjacent the contact surface 123A. Alternatively, as shown in FIG. 1B, the imaging housing 112B may be mounted such that signals from the imaging transducer 111B also pass through the transition member 115B. Alternatively, other imaging devices may be provided capable of generating signals that may be used to identify the target nerve 90 or other tissue of interest.

Optionally, the imaging element 110A, 110B may be movable relative to the housing 122A, 122B to change the direction of the axis “x”. For example, as shown, the imaging element 110A, 110B may be mounted to or adjacent the housing 122B, 122B by a mechanism that provides one or more degrees of freedom of movement of the imaging element 110A, 110B relative to the housing 122A, 122B and, consequently, relative to the transducers 124A, 124B. In the example shown, the imaging housing 112A, 112B may be mounted to the main housing 122A, 122B using a hinge 116A, 116B that allows the imaging element 110A, 110B to pivot relative to the housing 122A, 122B about a single axis, e.g., such that the imaging transducer 111A, 111B may be pivoted such that the axis “x” may be substantially parallel to the FUS ultrasound beam or may intersect the FUS ultrasound beam at an increasing acute angle, e.g., between about zero and ninety degrees (0-90°). In addition or alternatively, the entire imaging element 110A, 110B may be movable spatially relative to the housing 122A, 122B, e.g., in one or more directions within a plane parallel to the contact surface 123A, 123B (not shown). Alternatively, the imaging element 110A, 110B may be fixed relative to the housing 122A, 122B such that the intersection angle of the axis “x” and the FUS beam is fixed (even if the angles may be otherwise modified electronically as described elsewhere herein). Additional information regarding components that may be included in the focused ultrasound devices and systems herein may be found in International Publication No. WO 2022/266261, the entire disclosure of which is expressly incorporated by reference herein.

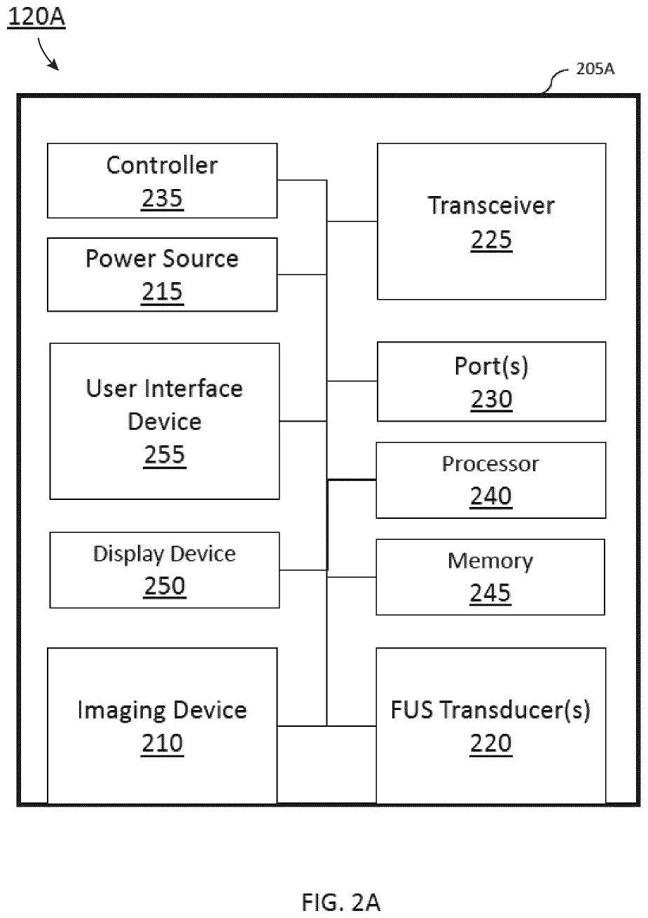

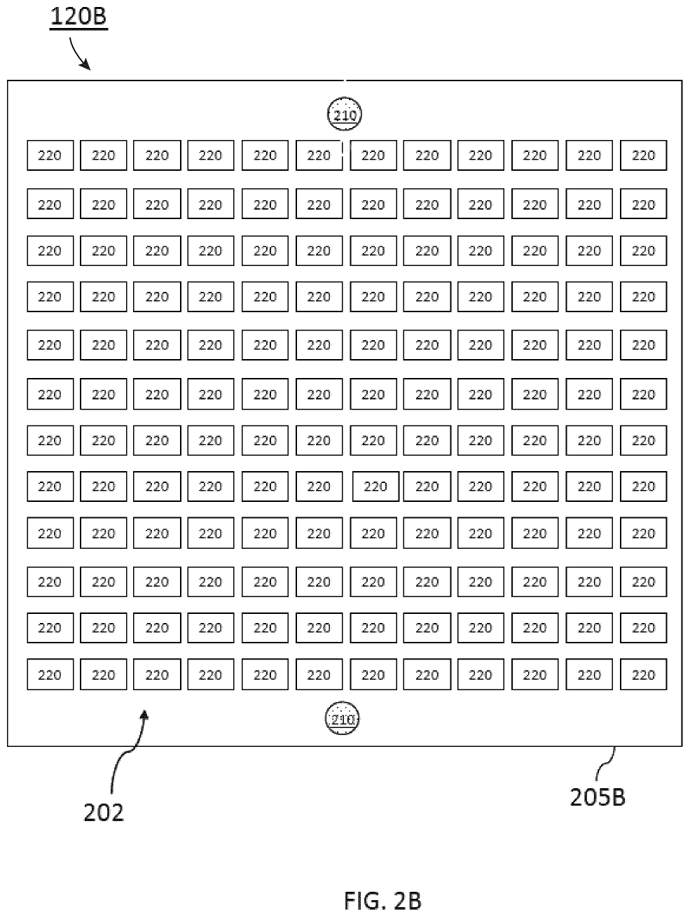

FIG. 2A is a block diagram of a set of components that may be included in a first exemplary FUS delivery device or system 120A, such as the system 100 shown in FIG. 1. The FUS delivery device 120A may include a housing 205A that houses one or more of an imaging device 210, one or more FUS transducer(s) 220 configured to emit focused ultrasonic energy into a subject toward a subject's target nerve (e.g., nerve 510), a power source 215, an optional transceiver 225, one or more ports 230, a controller 235, a processor 240, a memory 245, a display device 250, and/or a user interface device 255.

The power source 215 may be any source of electrical power for the FUS delivery device 120A including, but not limited to, a battery, a rechargeable battery, and/or a power cord configured to couple to an electrical main. Optional transceiver 225 may be any device configured to receive data and/or instructions from an external device (e.g., the processing device 140 and/or database 145) and/or communicate data (e.g., images, configuration settings, duty cycles, characteristics of FUS emission, etc.) to an external device. Optionally, the FUS delivery device 120A may include one or more ports 230 that may be configured, for example, to provide power to the FUS delivery device 120A and/or facilitate communication with one or more components of the FUS delivery device 120A. The controller 235 may be configured to control a configuration (e.g., focal zone geometry, FUS intensity, and/or frequency) and/or an operation (e.g., on/off) of, for example, the imaging device 210 and/or FUS transducer(s) 220. An operation of the controller 235 may be responsive to one or more instructions from the processor 240 and/or memory 245. The memory 245 may be configured to store, for example, one or more images obtained by the imaging device 210, information about a subject, and/or sets of instructions for calibrating the FUS transducer(s) 220 and/or setting one or more parameters for FUS emitted by the FUS transducer(s) 220.

The FUS transducer(s) 220 may be configured to project focused ultrasonic energy into the tissue of a subject toward one or more target nerve(s) and/or positions on, or proximate to, one or more target nerve(s) in accordance with, for example, one or more methods disclosed herein. In some examples, the FUS transducer(s) 220 may be configured to deliver focused low-intensity ultrasonic energy, such as ultrasonic energy with an exemplary intensity ranging between about zero and five hundred Watts per square centimeter (0-500 W/cm²) and an exemplary frequency within a range of about half to two Megahertz (0.5-2.0 MHz), e.g., about 1.5 MHz, to a target nerve and/or a target location on a nerve. Additionally, or alternatively, the FUS transducer(s) 220 may be configured to deliver ultrasonic energy at frequencies within a range of about one kilohertz and ten Megahertz (1 kHz-10 MHz) with an exemplary intensity ranging between about zero and five hundred Watts per square centimeter (0-500 W/cm²). Additionally, or alternatively, the FUS transducer(s) 220 may be configured to deliver relatively high-intensity ultrasonic energy with frequencies within a range between about one kilohertz and ten Megahertz (1 kHz-10 MHz) and an intensity within a range between about five hundred and two thousand Watts per square centimeter (500-2000 W/cm²).

The display device 250 may be configured, for example, to display an image obtained by the imaging device 210 and/or information (e.g., text and/or numbers) to a user. Exemplary display devices 250 include display screens and touch-sensitive display screens. The user interface device 255 may be configured to facilitate interaction between a user and the FUS delivery device 120A and/or a component of the system 100. Exemplary user interfaces 255 include, but are not limited to, buttons, keys, dials, and/pr microphones.

In some examples, one or more components of the system 100 and/or device 120A may be configured to recognize a target nerve, or group of target nerves, within imaged subject tissue by, for example, processing one or more images (e.g., a video) of the subject's tissue as imaged by, for example, the imaging device 110 and/or 210 to determine one or more characteristics of the subject's tissue and/or a target nerve within the subject's tissue. For example, one or more ultrasound images of the subject's tissue (e.g., an arm or knee) may be processed to identify a target nerve and/or one or more target locations therein.

FIG. 2B is a diagram of a second FUS delivery device 120B that includes an exemplary array 202 of one hundred and forty four (144) FUS transducers 220 arranged in a grid pattern within a housing 205B that also includes two optional imaging devices 210. In some examples, one or more of the FUS transducers 220 may be configured to rotate or move within or relative to the housing 205B, for example, to adjust the FUS focal zone geometry and/or position with subject tissue. The housing 205B may also house one or more additional components such as a power source 215, optional transceiver 225, one or more ports 230, controller 235, processor 240, memory 245, display device 250, and/or interface device 255, which are not shown in FIG. 2B. The second FUS delivery device 120B may be used, for example, to deliver FUS to multiple locations within a subject, e.g., as explained herein.

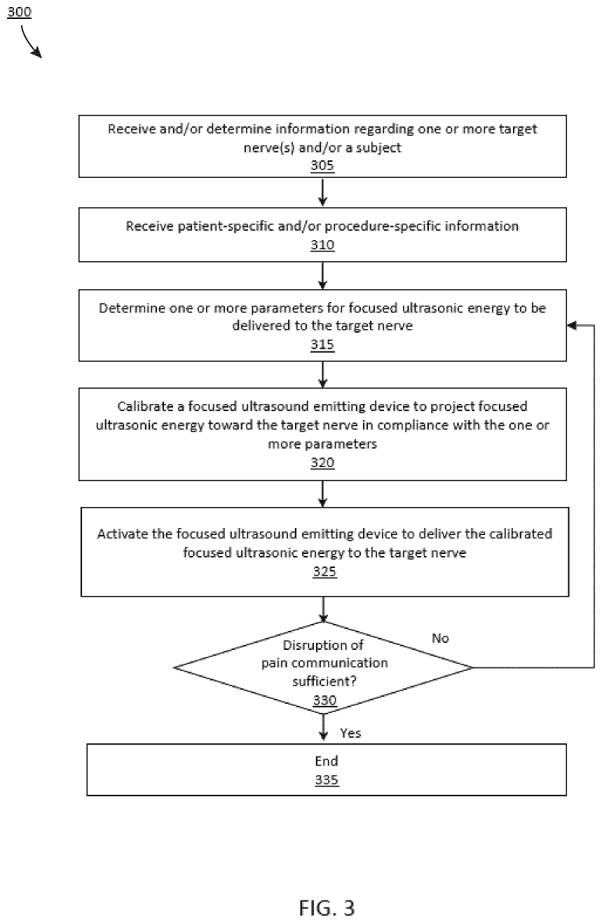

FIG. 3 is a flowchart illustrating an exemplary process 300 for delivering FUS to one or more target nerves within a subject. The process 300 may be executed using the system 100 and/or any of the systems and/or system components described herein.

In step 305, information regarding one or more target nerve(s) and/or a subject in which the one or more target nerve(s) resides may be received and/or determined, for example, by a processing device such as processing device 140 and/or processor 240. Exemplary information about the one or more target nerve(s) that may be received in step 305 includes, but is not limited to, one or more of size, shape, type, location, orientation, proximity to other nerves (target nerves or otherwise), whether treatment of two or more target nerves and/or two or more locations on the same target nerve are required and/or desired, and characteristics of surrounding tissue (e.g., sensitivity to FUS and/or type (e.g., muscle, adipose, etc.) of the tissue). Exemplary information that may be received about the subject includes, but is not limited to, one or of age, gender, weight, limbs, or body parts in communication with the target nerve, and/or characteristics of tissue (e.g., thickness, diameter, function, etc.) proximate to the target nerve. The information of step 305 may be received, for example, from an imaging device like imaging device 110 and/or 210, a database like database 145, and/or a user, for example, via user interface(s) 255 and/or 155.

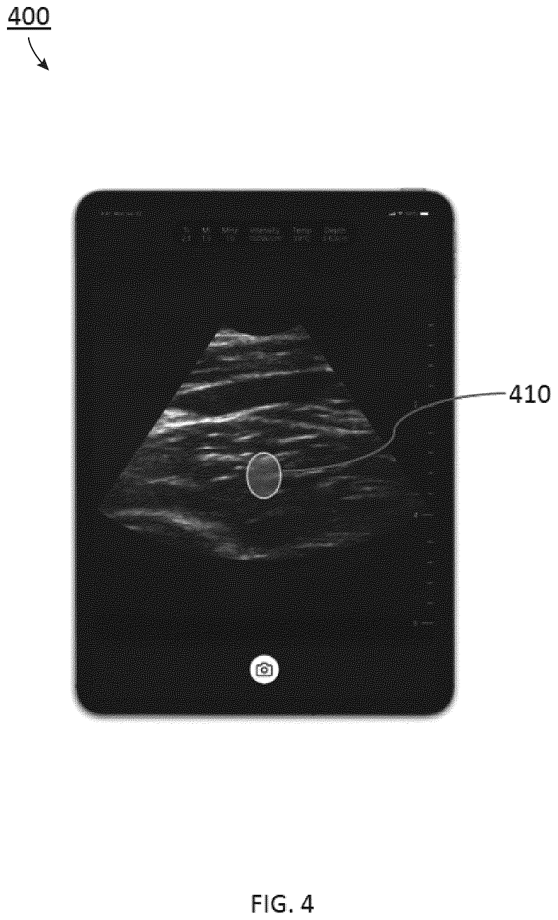

On some occasions, step 305 may be executed by providing an ultrasound image of the subject's tissue like image 400 of FIG. 4 to a display device such as display device 150 and/or 250 and receiving an indication of a notation 410 (in this case an oval) that a user manually adds into the image to, for example, mark a position of a target nerve, one or more geometrical aspects (e.g., diameter, width, length, etc.) of a desired FUS focal zone, and/or mark an area of the subject upon which FUS is to be directed. Additionally, or alternatively, execution of step 305 may include analysis (via, for example, image recognition software) of an image, such as image 400 shown in FIG. 4, by a processing device such as the processing device 140 and/or processor 240, for example, to detect, identify, resolve, and/or locate a target nerve and/or determine a size, shape and/or position of a FUS focal zone that may be used to treat the target nerve with FUS.

At times, additional information about the one or more target nerve(s) may be received and/or determined in step 305. Exemplary additional information that may be received includes, but is not limited to, whether the target nerve(s) is/are in an inflamed state and/or a level of sensitivity of the target nerve(s) (e.g., how responsive the subject is to the detection of pain stimuli by the target nerve). In some examples, step 305 may be executed once (e.g., prior to delivery of a short (e.g., 10-60 second) burst of FUS to a target nerve), as needed, and/or continuously over time to, for example, detect movement of a target nerve and/or facilitate accurate targeting of the FUS.

Optionally, in step 310, patient-specific and/or procedure-specific information may be received. Exemplary subject-specific information includes, but is not limited to, one or more of age, gender, size, body mass index, comorbidities (e.g., diabetes mellitus, neuropathy, etc.), a depth of a target nerve within the subject's tissue, and/or a type of tissue positioned between a FUS delivery device emitting FUS toward the target nerve and the target nerve. In an example where the subject has suffered a traumatic injury (e.g., car accident or gunshot wound), information about the traumatic injury and/or the state of the subject's health and/or medical condition (e.g., shock, level of consciousness, blood loss, etc.) may also be received in step 310. Exemplary procedure-specific information includes, but is not limited to, information about an injury the subject has suffered and/or a type of treatment procedure (e.g., surgery, dental drilling, bone setting, amputation, brachytherapy, etc.) the subject is expected to undergo and/or which nerves may contribute to sensations of pain the subject may experience during and/or after the procedure.

In step 315, one or more parameters for FUS to be delivered to the target nerve by one or more FUS delivery device(s), such as FUS transducer(s) 220 and/or FUS delivery device/system 120 shown in FIGS. 1-2, may be determined responsively, for example, to the information received and/or determined in steps 305 and 310. Exemplary parameters include, but are not limited to, one or more of intensity, frequency, and focal zone geometry (i.e., coverage area) for the FUS within the subject's tissue and/or on the target nerve and how many FUS delivery devices may be needed to adequately treat (e.g., cover an entire cross-sectional diameter) the target nerve. These parameters may be determined, for example, to achieve a desired duration (e.g., hours, days, months) of communication inhibition or “blockade” for pain sensations from the target nerve, a desired degree of neuromodulation of the target nerve, and/or a desired degree of nerve communication interruption (e.g., total, or partial blockade of pain sensations communicated by the target nerve). Additionally, or alternatively, the parameters of step 315 may be determined so that they induce a transient change to nerve structure that is expected to heal and/or regenerate over time so that, for example, an ability for the target nerve to sense pain and/or communicate sensations of pain to the central nervous system may be restored over time as a result of the neuroregeneration process. For example, the operator may identify a degree and/or time period of desired blockade of the target tissue, e.g., having a target period lasting hours, days, or months, as described elsewhere herein, and the controller (e.g., controller 235 in FIG. 2A) may automatically set parameters for the treatment to achieve the identified blockade.

In some examples, one or more parameters for the FUS to be delivered to the target nerve(s) may be set so that the focal zone geometry focuses the FUS on a region of the subject's tissue that completely covers and/or is larger than the target nerve(s). This may ensure, for example, that an entire cross-sectional area of each target nerve and/or each location of interest on a target nerve is modulated by the FUS. When an entire cross-sectional area of the target nerve(s) is/are modulated by the FUS energy, communication of pain sensations perceived by the target nerve(s) may be completely disrupted, which may serve to block any sensation of pain for the subject germinating from the treated target nerve(s). Often times, one or more of the parameters determined in step 315 may be selected and/or determined so that communication of pain information to the subject's central nervous system is inhibited without disruption of sensory or motor capability of the target nerve(s).

In step 320, the FUS delivery device may be calibrated to deliver or direct FUS toward the target nerve(s) in accordance with the one or more parameters for FUS determined in step 315. Once the FUS delivery device is in position on the subject's skin so that a projected or modeled FUS focal zone is expected to properly overlap with the target nerve(s), at step 325, the FUS delivery device may be activated (e.g., turned on or triggered) so that it emits FUS into the subject's body and modulates the target nerve(s). The FUS delivery device may emit FUS for a time period determined, in step 315, to be of sufficient duration to achieve a desired degree of modulation of the target nerve(s) that may sufficiently inhibit communication of pain information by the target nerve(s), thereby disrupting a sensation of pain from the target nerve(s) for the subject.

Optionally, in step 330, it may be determined whether a disruption of pain communication by the target nerve(s) is/are sufficient to completely and/or partially block the subject's sensation of pain germinating from the target nerve(s) and, if so, the process 300 may end at 335. If not, steps 315-330 may be repeated one or more times, as desired. Step 330 may be executed, for example, by receiving input from a user that the subject is not registering or experiencing pain in a portion of the body for which the target nerve(s) would ordinarily register pain. Optionally, the system may identify if the target nerve(s) has moved during delivery of the FUS. For example, if the system identifies that the nerve has moved in images being displayed and processed by the controller, e.g., if the subject moves and/or the delivery device is somehow displaced relative to the subject, the system may discontinue delivering the FUS. Once the delivery device is again stabilized, the controller may identify an additional period of delivery of FUS to complete a selected degree of modulation.

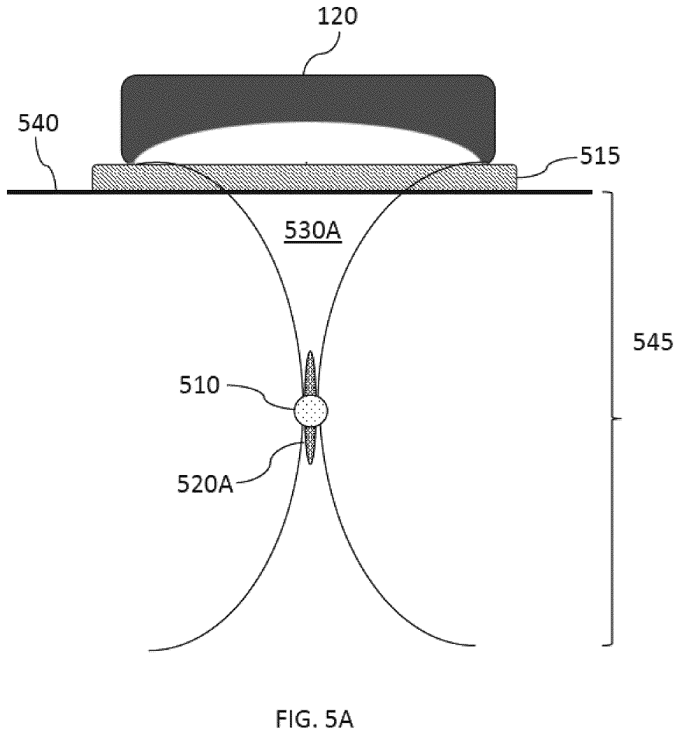

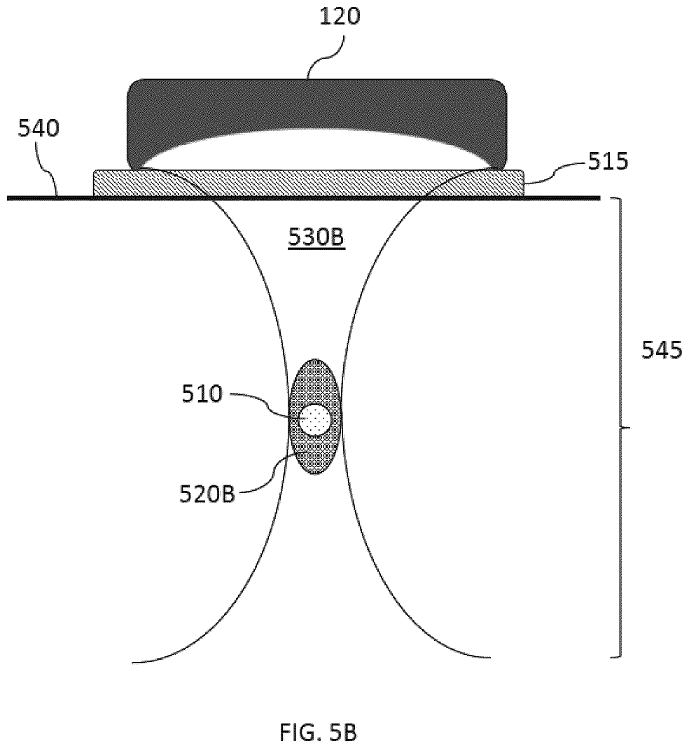

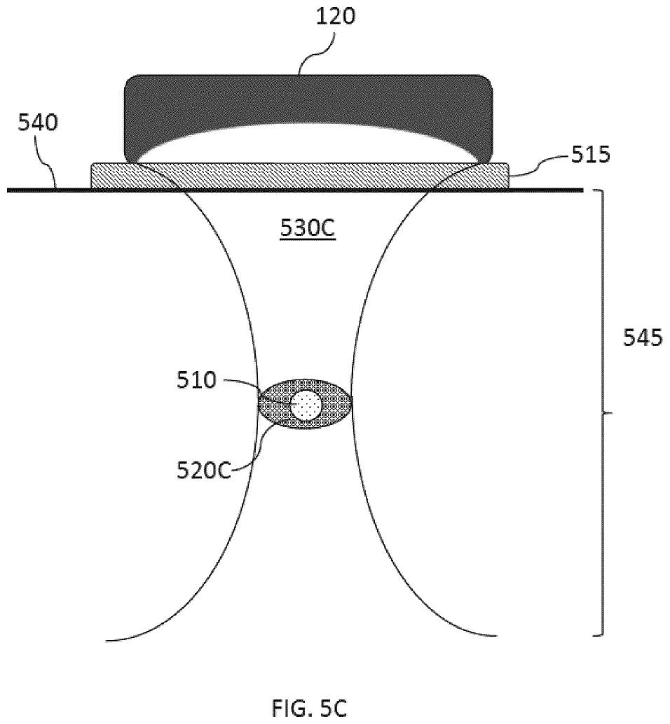

FIG. 5A is a two-dimensional schematic diagram showing a cross-section of a subject's tissue 545 (e.g., an arm, leg, shoulder, back, ankle, etc.) that includes a target nerve 510 with a FUS delivery device 120 positioned on a pad 515 that is interposed between the subject's epidermis 540 and FUS delivery device 120 as shown. FUS delivery device 120 may be configured to deliver a first beam of FUS 530A transcutaneously to target nerve 510 via a first FUS focal zone 520A (e.g., area of concentrated FUS) that has a shape of an elongated oval (i.e., narrower along a X-axis than a Y-axis (as oriented in the figure)) so that an area of concentrated, or focused, FUS intersects with a center of nerve 510 but does not cover an entirety of nerve 110 or any tissue to the left or right (as oriented in the figure) of target nerve 510.

The pad 515 may be configured, for example, to acoustically couple the delivery device 120 to the epidermis 540 and/or otherwise allow FUS to pass therethrough and adjust a position (e.g., depth) of a FUS focal zone, such as first FUS focal zone 520A, so that it is properly positioned relative to nerve 510. The pad 515 may be made, for example, from a transparent material with a medium (e.g., water, saline, and/or gel) contained therein. In some examples, a dimension (e.g., height, width, area, etc.) of pad 515 may be adjustable and/or configurable to, for example, properly position FUS delivery device 120 relative to the target nerve 510. In some examples, multiple pads 515 may be used to achieve a desired positioning of the FUS delivery device 120 relative to the nerve 510. In addition or alternatively, acoustic gel or other materials may be applied to one or more of the delivery device 120, pad 515, and/or epidermis 540, if desired to further acoustically couple the delivery device 120 to facilitate transmission of FUS to the target focal zone 520A.

FIG. 5B is a two-dimensional schematic diagram showing a cross-section of subject tissue 545 that includes target nerve 510 with a FUS delivery device 120 positioned on a pad 515, which is interposed between the subject's epidermis 540 and the FUS delivery device 120. In this example, the FUS delivery device 120 is arranged so that it is in a position directly above (as oriented in FIG. 5B) target nerve 510. The FUS delivery device 120 may be configured to deliver a second beam of FUS 530B transcutaneously to the target nerve 510 via a second FUS focal zone 520B. As may be seen in FIG. 5B, the second beam of FUS 530B is wider than the first beam of FUS 530A shown in FIG. 5A, and the second FUS focal zone 520B has a geometry that is oval shaped (i.e., narrower along a horizontal or X-axis than along a vertical or Y-axis (as oriented within the plane shown in FIG. 5B). The second FUS focal zone 520B is also wider along the X-axis than the first focal zone 520A so that an area of concentrated, or focused, FUS covers an entire cross-section of the target nerve 510 as shown. A focal zone geometry of this shape may be used, for example, when modulation of the target nerve 510 across an entire cross section of the nerve is desired.

FIG. 5C is a two-dimensional schematic diagram showing a cross-section of subject tissue 545 that includes target nerve 510 with a FUS delivery device 120 positioned on a pad 515, which is interposed between the subject's epidermis 540 and the FUS delivery device 120, wherein the FUS delivery device 120 is arranged so that it is in a position directly above (as oriented in FIG. 5C) the target nerve 510. In the example of FIG. 5C, the FUS delivery device 120 may be configured to deliver a third beam of FUS 530C transcutaneously to the target nerve 510 via a third FUS focal zone 520C. The third beam of FUS 530 C is wider than the second beam of FUS 530B shown in FIG. 5B, and the third FUS focal zone 520C has a geometry that is similar to the geometry of the second focal zone 520B but, is rotated by about ninety degrees (90°) so that the oval-shaped third FUS focal zone 520C is narrower along a vertical or Y-axis than along a horizontal or X-axis (as oriented within the plane shown in FIG. 5C). A focal zone geometry of this shape may be used, for example, when the target nerve is wider in the X-direction than in the Y-direction (e.g., has a flat or ribbon-shaped cross section).

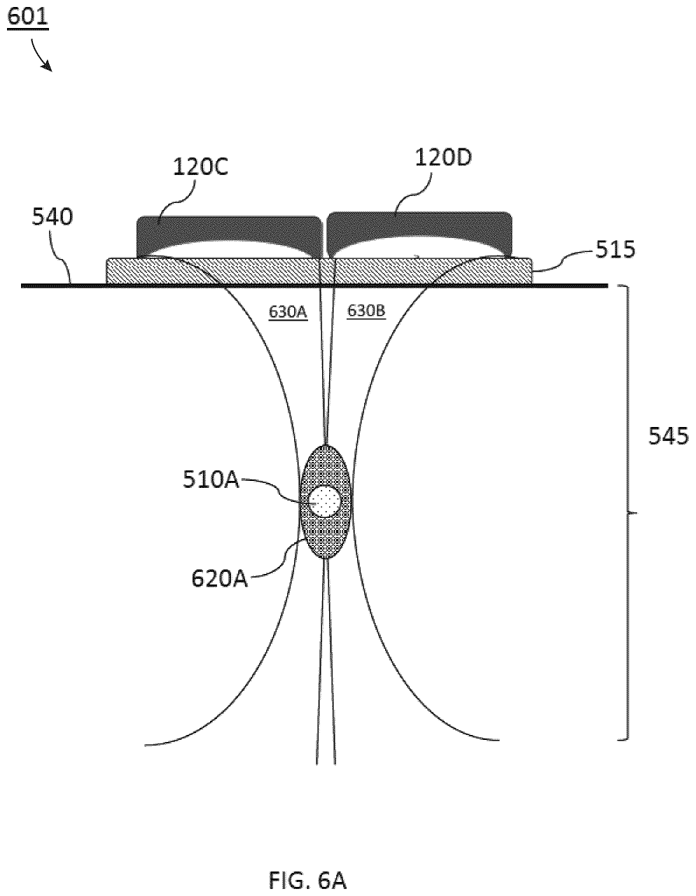

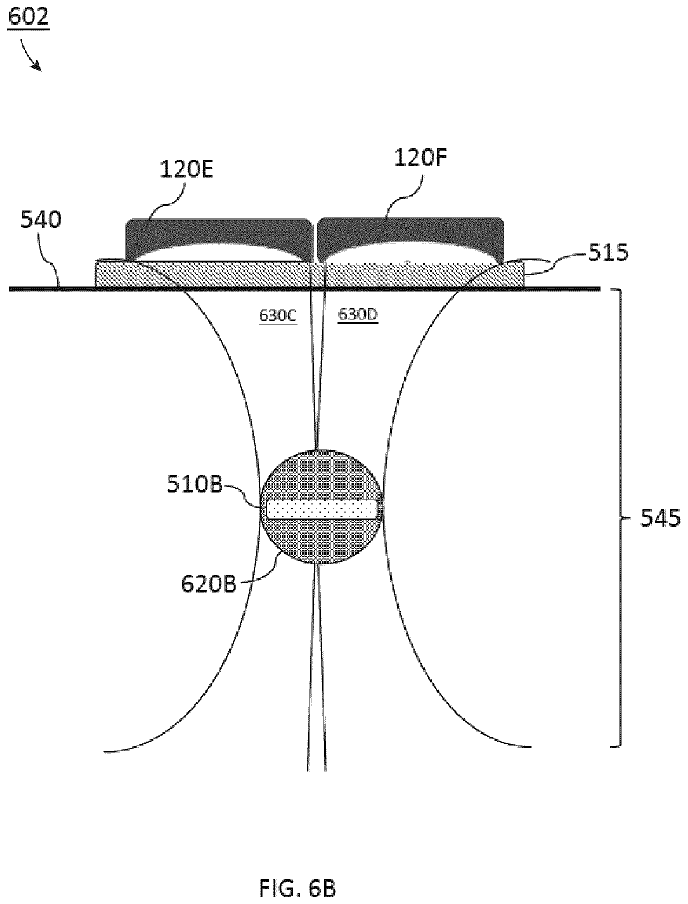

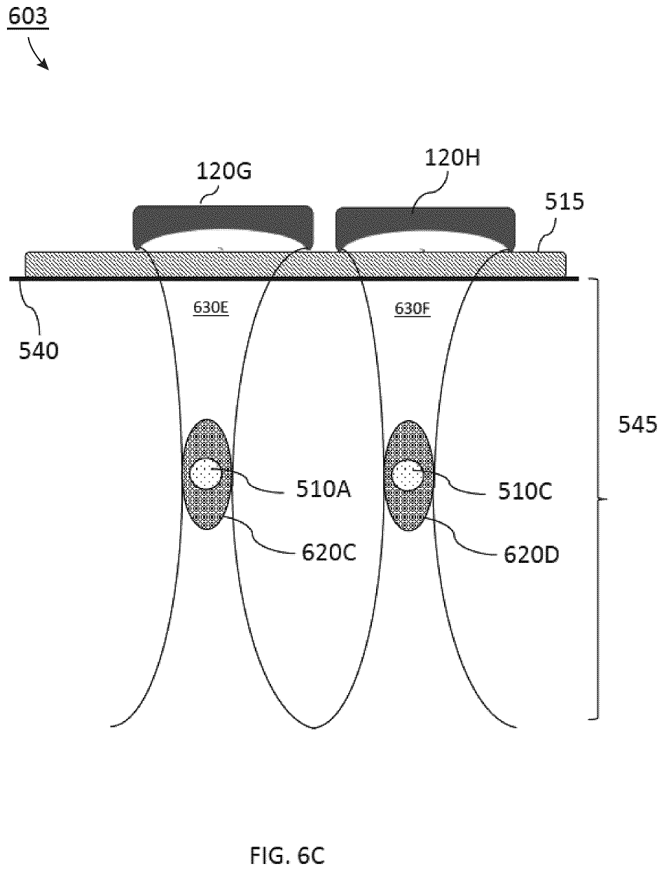

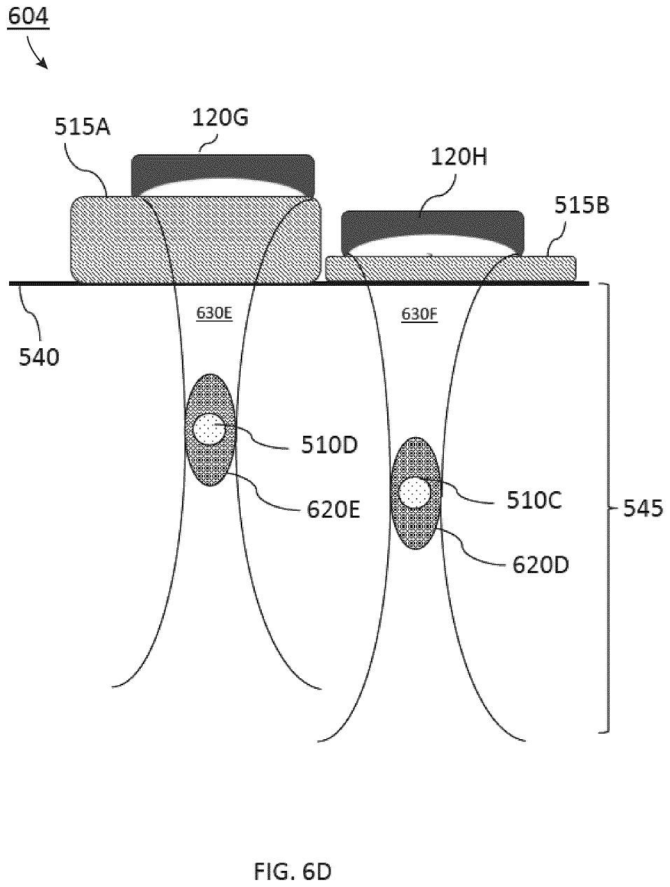

FIG. 6A is a two-dimensional schematic diagram showing a cross-section of subject tissue 545 that includes a first target nerve 510A with a first FUS delivery system 601 positioned on a pad 515, which is interposed between the subject's epidermis 540 and the first FUS delivery system 601, wherein the first FUS delivery system 601 is arranged in a position directly above (as oriented in FIG. 6A) the first target nerve 510A. The first FUS delivery system 601 includes a set of two FUS delivery devices including a first FUS delivery device 120C, which is configured to generate a first partial beam of FUS 630A, and a second FUS delivery device 120D configured to generate a second partial beam of FUS 630B. The first and second partial beams of FUS 630A and 630B combine proximate to the first target nerve 510A to form a first composite FUS focal zone 620A that exposes an entire cross section of the first target nerve 510A to FUS as shown. The first composite FUS focal zone 620A has a geometry that is oval shaped (i.e., narrower along a horizontal or X-axis than along a vertical or Y-axis (as within the plane shown in FIG. 6A) so that an area of concentrated, or focused, FUS covers an entire cross-section of the first target nerve 510A as shown. In some examples, the first and second FUS devices 120C and 120D may be configured, focused, and/or aligned so that the first and second partial beams of FUS 630A and 630B do not overlap one another but, instead, cooperate to deliver an approximately equal intensity of FUS across the first composite FUS focal zone 620A.

FIG. 6B is a two-dimensional schematic diagram showing a cross-section of subject tissue 545 that includes a relatively wide and thin target nerve 510B with a second FUS delivery system 602 positioned on a pad 515, which is interposed between the subject's epidermis 540 and the second FUS delivery system 602, with the second FUS delivery system 602 arranged on the pad 515 so that FUS may be projected through the subject's epidermis 540 toward the target nerve 510B. The second FUS delivery system 602 includes first and second delivery devices 120E and 120F, which are configured to generate a third portion of a composite wide beam of FUS 630C and a fourth portion of composite beam of FUS 630D, respectively, that together form a second composite FUS focal zone 620B that exposes an entire cross section of the target nerve 510B to FUS. The second composite FUS focal zone 620B has a geometry that is oval shaped (i.e., narrower along a vertical or Y-axis than along a horizontal or X-axis (as oriented within the plane shown in FIG. 6B) so that an area of concentrated, or focused, FUS covers an entire cross-section of the target nerve 510B as shown. Each of the first and second FUS delivery devices 120E and 120F of the FUS delivery system 602 may be configured, focused, and/or aligned so that beams of FUS 640C and 640D do not overlap one another but, instead, cooperate to deliver an approximately equal intensity of FUS across the second composite FUS focal zone 620B to the target nerve 510B.

FIG. 6C is a two-dimensional schematic diagram showing a third FUS delivery system 603 that may be used when, for example, treating two target nerves with FUS. The third FUS delivery system 603 includes first and second FUS delivery devices 120G, 120H positioned to delivery FUS to a first target nerve 510A and a second target nerve 510C, respectively. Both FUS delivery devices 120G and 120H are positioned on a pad 515, which is interposed between the subject's epidermis 540 and the third FUS delivery system 603. In the example shown in FIG. 6C, the first FUS delivery device 120G is configured to deliver a fifth beam of FUS 630E to the first target nerve 510A with a first FUS focal zone 620C being positioned around/through the first target nerve 510A as shown. In addition, the second FUS delivery device 120H is configured to deliver a second beam of FUS 630F to the second target nerve 510C with a second FUS focal zone 620D being positioned around/through the second target nerve 510C as shown.

FIG. 6D is a two-dimensional schematic diagram showing a fourth FUS delivery system 604 that may be used when, for example, treating two target nerves of different depths within patient tissue 545 with FUS. The FUS delivery system 604 includes first and second FUS delivery devices 120G, 120H positioned to deliver FUS to a first target nerve 510D and a second target nerve 510C, respectively. As shown in FIG. 6D, the first FUS delivery device 120G is positioned on a first pad 515A, which is interposed between the subject's epidermis 540 and the FUS delivery device 120G, and the second FUS delivery device 120H is positioned on a second pad 515B, which is interposed between the subject's epidermis 540 and the second FUS delivery device 120H. The first pad 515A is thicker than the second pad 515B so that a position of a first FUS focal zone 620E may be closer to the epidermis 540 to accommodate for a position of the first target nerve 510D, which is closer to the epidermis 540 than the second target nerve 510C.

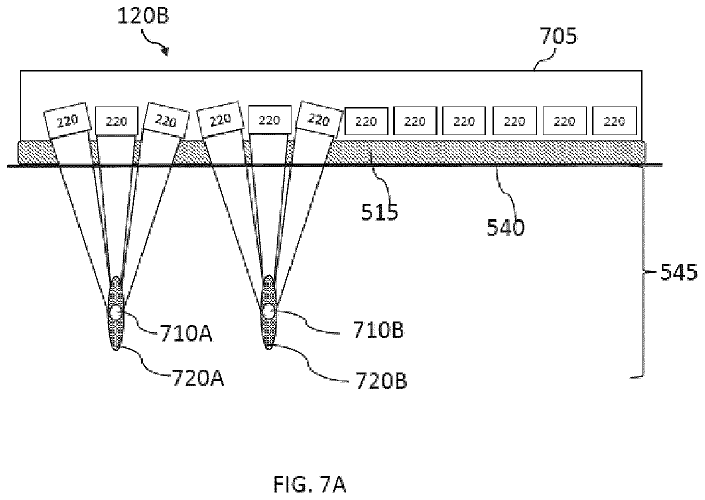

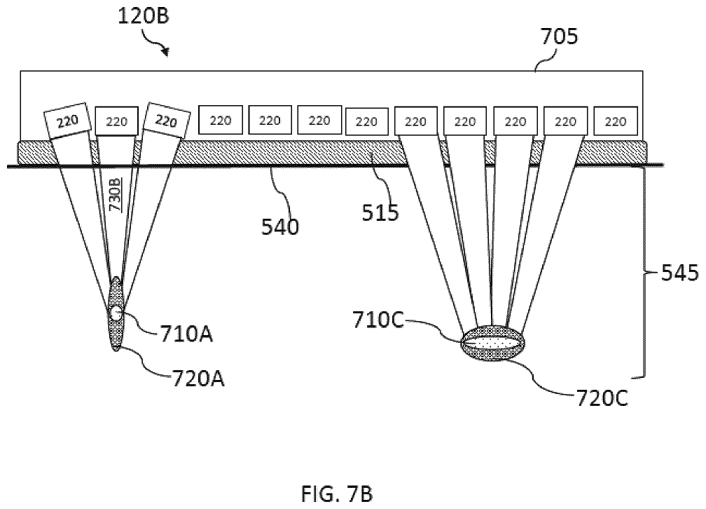

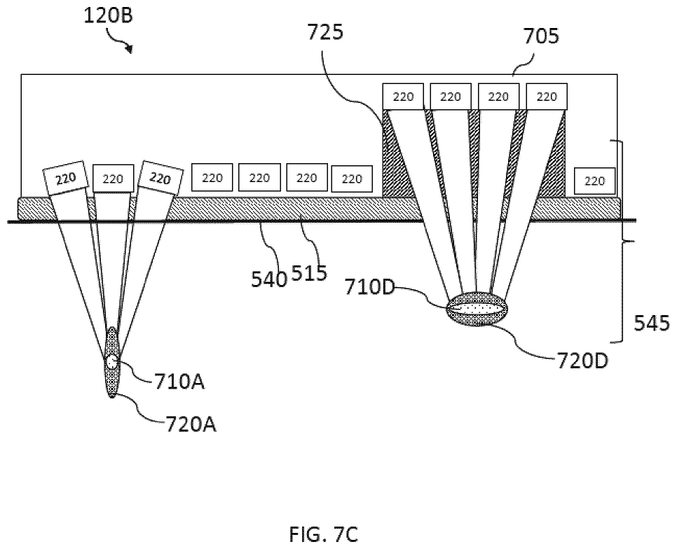

FIGS. 7A-7C are diagrams that illustrate different ways in which another FUS delivery device 120B may be used to modulate one or more target nerves that, in some cases, may be in different locations (e.g., along an X-, Y-, and/or Z-axis) and/or have different physical (e.g., size, shape, etc.) characteristics using one or more of the methods disclosed herein. The target nerves treated with FUS via the second FUS delivery device 120B may be located within subject tissue via, for example, examination and/or analysis of one or more images provided, for example, by the imaging device 210 shown in FIG. 2A. In particular, FIG. 7A provides a cross-sectional view of the second FUS delivery device 120B positioned on a pad 515, wherein the pad 515 is positioned on the subject's epidermis 540 above a first target nerve 710A and a second target nerve 710B positioned within the subject's tissue 545.

In the example of FIG. 7A, the first target nerve 710A is exposed to a first composite FUS focal zone 720A, which is generated by FUS emitted by the three leftmost (as oriented in the figure) FUS transducers 220 each emitting a FUS beam that cooperate to form first composite FUS focal zone 720A, e.g., in a manner similar to that described above with regard to FIGS. 6A and 6B. In addition, FIG. 7A illustrates that the second target nerve 710B is exposed to a second composite FUS focal zone 720B, which is generated by FUS emitted by a fourth, fifth, and sixth leftmost (as oriented in the figure) FUS transducers 220 each emitting a FUS beam that cooperate to form the second composite FUS focal zone 720B, e.g., also in a manner similar to that described above with regard to FIGS. 6A and 6B. In this way, both the first and second target nerves 710A and 710B may be treated with FUS at the same time and with the same device.

FIG. 7B provides a cross section view of a FUS delivery device 120B positioned on a pad 515, which is positioned on the subject's epidermis 540. The FUS delivery device 120B is arranged above a first target nerve 710A and a second target nerve 710C both of which are positioned within the subject's tissue 545. The second target nerve 710 has a wider, flatter, shape than the first target nerve 710 and the FUS delivery device 120B facilitates exposing that the entire cross section of the second target nerve 710C to FUS via four different beams of FUS emitted by eighth, ninth, tenth, and eleventh (counting from the left side) FUS transducers 220 that cooperate to form a second composite FUS focal zone 720C, e.g., in a manner similar to that described above with regard to FIGS. 6A and 6B as shown.

FIG. 7C provides a cross section view of a FUS delivery device 120B positioned on a pad 515, which is positioned on the subject's epidermis 540. The FUS delivery device 120B is arranged above a first target nerve 710A and a second target nerve 710D, which has a similar shape to the second target nerve 710C shown in FIG. 7B, but is closer to the subject's epidermis 540 than the second target nerve 710C. To account for this change in depth of the target nerve, eighth, ninth, tenth, and eleventh (counting from the left side) FUS transducers 220 are positioned on a secondary pad 725 to elevate them within the FUS device 120B so that a second combined FUS focal zone 720D penetrates to a proper depth within the subject's tissue 545 so that the second FUS focal zone 720D aligns with the second target nerve 710D, thereby exposing the entire cross-section of the second target nerve 710D to FUS.

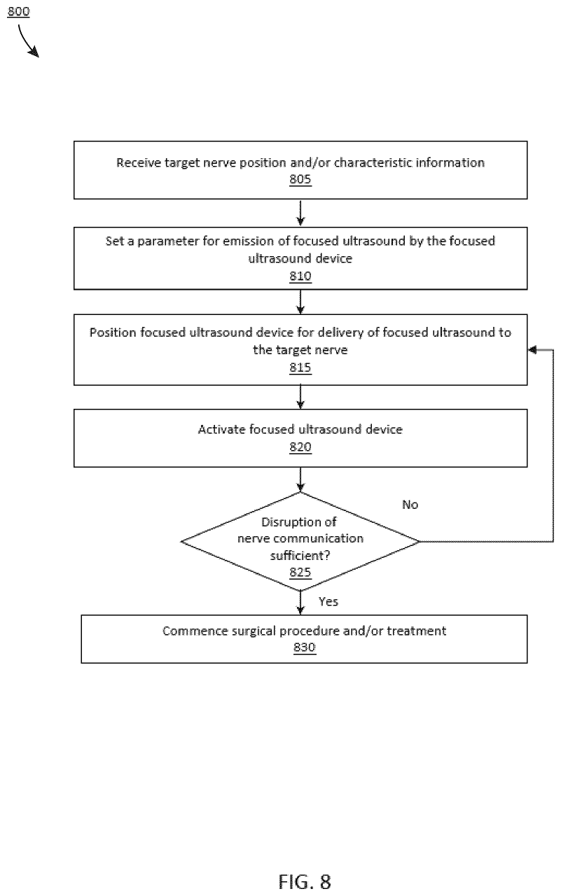

FIG. 8 is a flowchart illustrating a process 800 for treating a target nerve (e.g., target nerve 510) of a subject using FUS. The process 800 may be executed, for example, by a clinician using the system 100 shown in FIG. 1 or any of the systems or system components described herein. In some examples, the process 800, or portions thereof, may be executed by the clinician before an event that is expected to cause the subject acute pain such as surgery or execution of a medical treatment (e.g., drilling out a dental cavity or repositioning a broken bone) upon the subject.

Optionally, in step 805, information (e.g., size, type, location, orientation, etc.) regarding a target nerve may be received. The information may be received, for example, from a user via user interface 255 and/or 155, an imaging device such as imaging device 110 and/or 210, a database such as database 145, a processing device such as processing device 140, a transceiver such as transceiver 225, and/or a display device such as display device 150 and/or 250 shown in FIGS. 1 and 2A. At step 810, the clinician may set or input one or more parameters (e.g., intensity, frequency, and/or focal zone geometry) for the FUS to be emitted by the FUS delivery device, for example, by interaction with a user interface device communicatively coupled to the FUS delivery device, such as user interface 155 and/or 255, shown in FIGS. 1 and 2A. In some examples, one or more parameters may be determined, for example, via step 315 of process 300 discussed elsewhere herein.

At step 815, the clinician may then position a FUS delivery device, such as FUS transducer(s) 220 and/or FUS delivery device/system 120, shown in FIGS. 1 and 2A, so that the delivery device may deliver FUS to the target nerve. Once in position, at step 820, the FUS delivery device may be activated, for example, to modulate the target nerve thereby inhibiting the target nerve ability to communicate sensory information that may be interpreted as pain.

Optionally, in step 825, it may be determined whether a disruption of sensory communication by the target nerve is sufficient to completely and/or partially block the subject's sensation of pain by the target nerve and, if so, execution of the surgical procedure and/or medical treatment may commence. If not, steps 815-825 may be repeated. Step 825 may be executed, for example, by the clinician testing a reaction of the subject to stimulation of a region of his or her body corresponding to the target nerve.

While the invention is susceptible to various modifications, and alternative forms, specific examples thereof have been shown in the drawings and are herein described in detail. It should be understood, however, that the invention is not to be limited to the particular forms or methods disclosed, but to the contrary, the invention is to cover all modifications, equivalents and alternatives falling within the scope of the appended claims.

1. A system for modulating peripheral sensory nerves and/or peripheral sensory nerve fibers in a subject's body, comprising:
an imaging device configured to identify a target nerve within the subject's body;
a housing including a surface configured for placement against the subject's skin;
one or more transducer elements carried by the housing configured to deliver focused ultrasound from the surface into the subject's body; and
a controller coupled to the imaging device to identify the target nerve and coupled to the one or more transducer elements to control delivery of the focused ultrasound to the target nerve.

2-21. (canceled)

22. The system of claim 1, wherein the controller is configured to:
a) analyze information regarding the target nerve;
b) determine one or more parameters of the focused ultrasound based at least in part on a target degree of neuromodulation of the target nerve;
c) calibrate the one or more transducers to deliver focused ultrasound based on the one or more parameters; and
d) activate the one or more transducers to deliver the focused ultrasound to the target nerve.

23. The system of claim 22, wherein the controller is further configured to:
e) deactivate the one or more transducers to discontinue delivery of the focused ultrasound;
f) determine whether the target degree of neuromodulation has been achieved; and
if additional focused ultrasound is needed to achieve the target degree of neuromodulation, repeating steps b) to e).

24. The system of claim 22, wherein analyzing information regarding the target nerve comprises analyzing signals from the imaging device to identify the location of the target nerve relative to the one or more transducers.

25. The system of claim 22, wherein analyzing information regarding the target nerve comprises analyzing signals from the imaging device to identify whether the target nerve is in an inflamed state.

26. The system of claim 22, wherein analyzing information regarding the target nerve comprises analyzing signals from the imaging device to identify sensitivity of the target nerve.

27. The system of claim 22, further comprising, while the one or more transducers are activated, the controller is further configured to:
e) analyze signals from the imaging device to identify whether the target nerve moves relative to the one or more transducers; and
f) if the target nerve has moved, deactivate the one or more transducers to discontinue delivery of the focused ultrasound.

28. The system of claim 22, further comprising:
a display coupled to the controller to present images of the subject's body including the target nerve; and
a user interface configured to allow an operator of the system to identify a target focal zone on the display relative to a target nerve,
wherein the controller is configured to automatically determine the one or more parameters and calibrate the one or more transducers based at least in part on the target focal zone identified on the display.

29. The system of claim 1, wherein the housing and the imaging device are mounted to an arm fixed to a structure that may be positioned adjacent the subject to allow the position of the one or more transducer elements and imaging device to be adjusted by moving the arm.

30-31. (canceled)

32. The system of claim 22, further comprising:
a display coupled to the controller to present images of the subject's body including the target nerve; and
a user interface configured to allow an operator of the system to adjust a location of a target focal zone on the display relative to a target nerve,
wherein the controller is configured to automatically determine the one or more parameters and calibrate the one or more transducers based at least in part on the target focal zone identified on the display.

33. A method for modulating peripheral sensory nerves and/or peripheral sensory nerve fibers in a subject's body, comprising:
placing a surface of a housing against the subject's skin to position one or more transducer elements carried by the housing to deliver focused ultrasound from the surface into the subject's body;
acquiring images of locations within the subject's body using an imaging device mounted on the housing;
providing a controller configured to:
analyze information regarding the target nerve based at least in part on images acquired by the imaging device;
determine one or more parameters of the focused ultrasound based at least in part on a target degree of neuromodulation of the target nerve;
calibrate the one or more transducers to deliver focused ultrasound based on the one or more parameters; and
activate the one or more transducers to deliver the focused ultrasound to the target nerve.

34. The method of claim 33, further comprising identifying a target focal zone on a display relative to a target nerve presented on the display, and wherein the controller determines the one or more parameters and calibrates the one or more transducers based at least in part on the target focal zone identified on the display.

35. The method of claim 33, further comprising adjusting a location of a target focal zone on the display relative to a target nerve presented on the display, and wherein the controller determines the one or more parameters and calibrates the one or more transducers based at least in part on the location identified on the display.

36. The method of claim 33, further comprising setting a desired degree of blockade, and wherein the controller determines the one or more parameters and calibrates the one or more transducers based at least in part on the desired degree of blockade.

37. The method of claim 36, wherein the desired degree of blockade is within a range of about one to twenty four hours, one to six days, or one to twelve weeks.

38. The method of claim 33, wherein the controller is further configured to:
deactivate the one or more transducers to discontinue delivery of the focused ultrasound;
determine whether the target degree of neuromodulation has been achieved; and
if additional focused ultrasound is needed to achieve the target degree of neuromodulation, repeating the determining, calibrating, and activating steps to deliver additional focused ultrasound to the target nerve.

39. The method of claim 33, wherein analyzing information regarding the target nerve comprises analyzing signals from the imaging device to identify the location of the target nerve relative to the one or more transducers.

40. The method of claim 33, wherein analyzing information regarding the target nerve comprises analyzing signals from the imaging device to identify whether the target nerve is in an inflamed state.

41. A system for modulating peripheral sensory nerves and/or peripheral sensory nerve fibers in a subject's body, comprising:
a housing including a surface configured for placement against the subject's skin;
one or more transducer elements carried by the housing configured to deliver focused ultrasound from the surface into the subject's body;
an imaging device mounted on the housing adjacent the surface and configured to acquire images within the subject's body; and
a controller coupled to the imaging device and the one or more transducer elements, wherein the controller is configured to:
a) analyze information regarding a target nerve based at least in part on images acquired by the imaging device;
b) determine one or more parameters of the focused ultrasound based at least in part on a target degree of neuromodulation of the target nerve;
c) calibrate the one or more transducers to deliver focused ultrasound based on the one or more parameters; and
d) activate the one or more transducers to deliver the focused ultrasound to the target nerve.

42. The system of claim 41, wherein the controller is further configured to:
e) deactivate the one or more transducers to discontinue delivery of the focused ultrasound;
f) determine whether the target degree of neuromodulation has been achieved; and
if additional focused ultrasound is needed to achieve the target degree of neuromodulation, repeating steps b) to e).

43-73. (canceled)
